(19) United States
(12) Patent Application Publication
(10) Pub. No.: US 2009/0012486 A1
(43) Pub. Date: Jan. 8, 2009

(54) Process for Production of a Water-Absorbing Material

(75) Inventors: Ulrich Riegel, Landstuhl (DE); Thomas Daniel, Waldsee (DE); Stefan Bruhns, Mannheim (DE); Mark Elliott, Ludwigshafen (DE); Dieter Hermeling, Boehl-Iggelheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/159,298

(22) PCT Filed: Dec. 19, 2006
(86) PCT No.: PCT/EP2006/069892

§ 371 Date: Jun. 26, 2008

(30) Foreign Application Priority Data
Dec. 28, 2005 (EP) 05028545.1

(51) Int. Cl. A61F 13/15; B05D 7/00; C09K 3/00

(52) U.S. Cl. 604/358; 427/213; 252/194

(57) ABSTRACT

The present invention relates to a process for producing a water-absorbing material comprising the step of spray-coating water-absorbing polymeric particles with at least one non-reactive coating agent in a continuous process in a fluidized bed reactor in the range from 0° C. to 150° C., with the proviso that the non-reactive coating agents do not comprise an elastic film-forming polymer, the water-absorbing material obtainable by this process, the use of the water-absorbing material in hygiene articles and packaging material and hygiene articles comprising this material.

The present application relates to a process for producing a water-absorbing material having a functional shell and a water-absorbing material received according to this process, the use of the water-absorbing material in hygiene articles and packaging material and hygiene articles comprising this material.

An important component of disposable absorbent articles such as diapers is an absorbent core structure comprising water-absorbing polymers, typically hydrogel-forming water-absorbing polymers, also referred to as absorbent gelling material, AGM, or super-absorbent polymers, or SAP's. This polymer material ensures that large amounts of bodily fluids, e.g. urine, can be absorbed by the article during its use and locked away, thus providing low rewet and good skin dryness.

Especially useful water-absorbing polymers or SAP's are often made by initially polymerizing unsaturated carboxylic acids or derivatives thereof, such as acrylic acid, alkali metal (e.g., sodium and/or potassium) or ammonium salts of acrylic acid, alkyl acrylates, and the like in the presence of relatively small amounts of di- or poly-functional monomers such as N,N′-methylenebisacrylamide, trimethylolpropane triacrylate, ethylene glycol di(meth)acrylate, or triallylamine. The di- or poly-functional monomer materials serve to lightly cross-link the polymer chains thereby rendering them water-insoluble, yet water-absorbing. These lightly crosslinked absorbent polymers contain a multiplicity of carboxylate groups attached to the polymer backbone. It is generally believed that the neutralized carboxylate groups generate an osmotic driving force for the absorption of body fluids by the crosslinked polymer network. In addition, the polymer particles are often treated as to form a surface cross-linked layer on the outer surface in order to improve their properties in particular for application in baby diapers and adult hygiene articles.

Water-absorbing (hydrogel-forming) polymers useful as absorbents in absorbent members and articles such as disposable diapers need to have adequately high absorption capacity, as well as adequately high gel strength. Absorption capacity needs to be sufficiently high to enable the absorbent polymer to absorb significant amounts of the aqueous body fluids encountered during use of the absorbent article. Together with other properties of the gel, gel strength relates to the tendency of the swollen polymer particles to resist deformation under an applied stress. The gel strength needs to be high enough in the absorbent member or article so that the particles do not deform and fill the capillary void spaces to an unacceptable degree causing so-called gel blocking. This gel-blocking inhibits the rate of fluid uptake or the fluid distribution, i.e. once gel-blocking occurs, it can substantially impede the distribution of fluids to relatively dry zones or regions in the absorbent article and leakage from the absorbent article can take place well before the water-absorbing polymer particles are fully saturated or before the fluid can diffuse or wick past the “gel blocking” particles into the rest of the absorbent article. Thus, it is important that the water-absorbing polymers (when incorporated in an absorbent structure or article) maintain a high wet-porosity and have a high resistance against deformation thus yielding high permeability for fluid transport through the swollen gel bed.

Absorbent polymers with relatively high permeability can be made by increasing the level of internal crosslinking or surface crosslinking, which increases the resistance of the swollen gel against deformation by an external pressure such as the pressure caused by the wearer, but this typically also reduces the absorbent capacity of the gel which is undesirable. It is a significant draw back of this conventional approach that the absorbent capacity has to be sacrificed in order to gain permeability. The lower absorbent capacity must be compensated by a higher dosage of the absorbent polymer in hygiene articles, which for example leads to difficulties with the core integrity of a diaper during wear. Hence, special, technically challenging and expensive fixation technologies are required to overcome this issue in addition to the higher costs that are incurred because of the required higher absorbent polymer dosing level.

Inorganic powder coatings have also been described in the art (i.e. WO 02/060983) to improve the permeability of the absorbent polymer without reducing its capacity. Coatings with multivalent metal salts (i.e. WO 05/080479) or polycationic polymers (i.e. WO 04/024816) have also been described as useful to achieve this purpose. Application of these coatings is usually done via blending processes known to someone skilled in the art. In both cases it is however observed that the homogeneity of the absorbent polymer is not very consistent as these coatings typically cannot be homogenized across the particle surfaces of the absorbent polymer particles as these coatings show little or no diffusibility on the particle surface. Hence, the homogeneity realized during mixing and coating is reflected by fluctuating performance in each batch of absorbent polymer modified by such process. In addition when absorbent polymer particles bearing a shell of inorganic powder on its surfaces are subject to pneumatic conveying or mechanical transport it is often observed that the inorganic powder is stripped from the surface leading to more inhomogeneity and less consistent product performance. To overcome this problem it has been suggested in the literature to use a Polyol as dedusting agent (i.e. PCT/EP2005/011073 or a dendritic polymer (i.e. WO 05/061014, PCT/EP05/003009) which are all incorporated herein expressly by reference. Typically such polyol or polymer can be sprayed together with the inorganic powder onto the absorbent polymer's surface in order to provide an effective means of fixation for the inorganic powder but the homogeneity is still insufficient.

However, the application of such coatings takes place in the surface-coating step, which does not allow the use of heat-sensitive coatings and also the homogeneity and the resulting product performance is not optimized. If such coating is added in a separate mixer after the surface-coating step, homogeneity of the product is typically poor.

EP-A 0 703 265 teaches the treatment of hydrogel with film-forming polymers such as acrylic/methacrylic acid dispersions in a batch reactor to produce abrasion-resistant absorbents.

The present invention thus has for its objective to provide a process for producing a advantageous modification of the surface yielding water-absorbing material with a very homogeneous shell and highly consistent product properties over a long period.

We have found that this objective is achieved by a process comprising the step of spray-coating water-absorbing polymeric particles with at least one non-reactive coating agent in a continuous process in a fluidized bed reactor in the range from 0° C. to 150° C., with the proviso that the non-reactive coating agents do not comprise an elastic film-forming polymer.

An object of the invention is a process for producing a water-absorbing material comprising the step of spray-coating water-absorbing polymeric particles with at least one non-reactive coating agent in a continuous process in a fluidized bed reactor in the range from 0° C. to 150° C., wherein the non-reactive coating agent is selected from the group consisting of water-insoluble inorganic powders, water-soluble multivalent metal salts, polycationic polymers, sawdust and binding agents.

It will be appreciated that the herein above identified and the herein below still to be described features of the subject matter of the invention are utilizable not only in the particular combination that is specified but also in other combinations without leaving the realm of the invention.

Inert gases within the realm of this application are materials which are in gaseous form under the respective reaction conditions and which, under these conditions, do not have an oxidizing effect on the constituents of the reaction mixture or on the polymer, and also mixtures of these gases. Useful inert gases include for example nitrogen, carbon dioxide or argon, and nitrogen is preferred.

Useful for the purposes of the present invention are in principle all particulate water-absorbing polymers known to one skilled in the art from superabsorbent literature for example as described in Modern Superabsorbent Polymer Technology, F. L. Buchholz, A. T. Graham, Wiley 1998. The water-absorbing polymeric particles are preferably spherical water-absorbing polymeric particles of the kind typically obtained from inverse phase suspension polymerizations; they can also be optionally agglomerated at least to some extent to form larger irregular particles. But most particular preference is given to commercially available irregularly shaped particles of the kind obtainable by current state of the art production processes as is more particularly described hereinbelow by way of example.

The polymeric particles that are coated according to the present invention are preferably polymeric particles obtainable by polymerization of a monomer solution comprising
i) at least one ethylenically unsaturated acid-functional monomer,
ii) at least one crosslinker,
iii) if appropriate one or more ethylenically and/or allylically unsaturated monomers copolymerizable with i) and
iv) if appropriate one or more water-soluble polymers onto which the monomers i), ii) and if appropriate iii) can be at least partially grafted,
   wherein the base polymer obtained thereby is dried, classified and if appropriate is subsequently treated with
v) at least one post-crosslinker
   before being dried and thermally post-crosslinked (ie. surface crosslinked).

Useful monomers i) include for example ethylenically unsaturated carboxylic acids, such as acrylic acid, methacrylic acid, maleic acid, fumaric acid, tricarboxy ethylene and itaconic acid, or derivatives thereof, such as acrylamide, methacrylamide, acrylic esters and methacrylic esters. Acrylic acid and methacrylic acid are particularly preferred monomers. Acrylic acid is most preferable.

The water-absorbing polymers to be used according to the present invention are typically crosslinked, i.e., the polymerization is carried out in the presence of compounds having two or more polymerizable groups which can be free-radically copolymerized into the polymer network. Useful crosslinkers ii) include for example ethylene glycol dimethacrylate, diethylene glycol diacrylate, allyl methacrylate, trimethylolpropane triacrylate, triallylamine, tetraallyloxyethane as described in EP-A 530 438, di- and triacrylates as described in EP-A 547 847, EP-A 559 476, EP-A 632 068, WO 93/21237, WO 03/104299, WO 03/104300, WO 03/104301 and in DE-A 103 31 450, mixed acrylates which, as well as acrylate groups, comprise further ethylenically unsaturated groups, as described in DE-A 103 31 456 and DE-A 103 55 401, or crosslinker mixtures as described for example in DE-A 195 43 368, DE-A 196 46 484, WO 90/15830 and WO 02/32962.

Useful crosslinkers ii) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP-A 343 427. Useful crosslinkers ii) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process of the present invention preferably utilizes di(meth)acrylates of polyethylene glycols, the polyethylene glycol used having a molecular weight between 300 g/mole and 1000 g/mole.

Useful crosslinkers ii) are di- and triacrylates of altogether 1- to 100-tuply ethoxylated glycerol, trimethylolpropane, trimethylolethane, pentaerythritol, sorbitol, erythritol or similar two or more OH-groups bearing polyols. Also the respective Michael-condensation products that may be formed from these di- or triacrylates during the course of their synthesis are useful crosslinkers ii) by themselves or as part of a cross-linker mixture.

However, particularly advantageous crosslinkers ii) are di- and triacrylates of altogether 3- to 15-tuply ethoxylated glycerol, of altogether 3- to 15-tuply ethoxylated trimethylolpropane, especially di- and triacrylates of altogether 3-tuply ethoxylated glycerol or of altogether 3-tuply ethoxylated trimethylolpropane, of 3-tuply propoxylated glycerol, of 3-tuply propoxylated trimethylolpropane, and also of altogether 3-tuply mixedly ethoxylated or propoxylated glycerol, of altogether 3-tuply mixedly ethoxylated or propoxylated trimethylolpropane, of altogether 15-tuply ethoxylated glycerol, of altogether 15-tuply ethoxylated trimethylolpropane, of altogether 40-tuply ethoxylated glycerol and also of altogether 40-tuply ethoxylated trimethylolpropane. Where n-tuply ethoxylated means that n mols of ethylene oxide are reacted to one mole of the respective polyol with n being an integer number larger than 0.

Very particularly preferred for use as crosslinkers ii) are diacrylated, dimethacrylated, triacrylated or trimethacrylated multiply ethoxylated and/or propoxylated glycerols as described for example in prior PCT application WO 03/104 301. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred. These are notable for particularly low residual levels in the water-absorbing polymer (typically below 10 ppm) and the aqueous extracts of water-absorbing polymers produced therewith have an almost unchanged surface tension compared with water at the same temperature (typically not less than 0.068 N/m).

Examples of ethylenically unsaturated monomers iii) which are copolymerizable with the monomers i) are acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers iv) include polyvinyl alcohol, polyvinylpyrrolidone, starch, starch derivatives, polyglycols, polyacrylic acids, polyvinylamine or polyallylamine, partially hydrolysed polyvinylformamide or polyvinylacetamide, preferably polyvinyl alcohol and starch.

Preference is given to water-absorbing polymeric particles whose base polymer is lightly crosslinked. The light degree of crosslinking is reflected in the high CRC value and also in the fraction of extractables.

The crosslinker is preferably used (depending on its molecular weight and its exact composition) in such amounts that the base polymers produced have a CRC between 20 and 60 g/g when their particle size is between 150 and 850 μm and the 16 h extractables fraction is not more than 25% by weight. The CRC is preferably between 30 and 45 g/g, more preferably between 33 and 40 g/g.

Particular preference is given to base polymers having a 16 h extractables fraction of not more than 20% by weight, preferably not more than 15% by weight, even more preferably not more than 10% by weight and most preferably not more than 7% by weight and whose CRC values are within the preferred ranges that are described above.

The preparation of a suitable base polymer and also further useful hydrophilic ethylenically unsaturated monomers i) are described in DE-A 199 41 423, EP-A 686 650, WO 01/45758 and WO 03/14300.

The reaction is preferably carried out in a kneader as described for example in WO 01/38402, or on a belt reactor as described for example in EP-A 955 086.

It is further possible to use any conventional inverse suspension polymerization process. If appropriate, the fraction of crosslinker can be greatly reduced or completely omitted in such an inverse suspension polymerization process, since self-crosslinking occurs in such processes under certain conditions known to one skilled in the art.

It is further possible to make base polymers using any desired spray polymerization process.

The acid groups of the base polymers obtained are preferably 30-100 mol %, more preferably 65-90 mol % and most preferably 67-80 mol % neutralized, for which the customary neutralizing agents can be used, for example ammonia, or amines, such as ethanolamine, diethanolamine, triethanolamine or dimethylaminoethanolamine, preferably alkali metal hydroxides, alkali metal oxides, alkali metal carbonates or alkali metal bicarbonates and also mixtures thereof, in which case sodium and potassium are particularly preferred as alkali metals, but most preferred is sodium hydroxide, sodium carbonate or sodium bicarbonate and also mixtures thereof. Typically, neutralization is achieved by admixing the neutralizing agent as an aqueous solution or as an aqueous dispersion or else preferably as a molten or as a solid material.

Neutralization can be carried out after polymerization, at the base polymer stage. But it is also possible to neutralize up to 40 mol %, preferably from 10 to 30 mol % and more preferably from 15 to 25 mol % of the acid groups before polymerization by adding a portion of the neutralizing agent to the monomer solution and to set the desired final degree of neutralization only after polymerization, at the base polymer stage. The monomer solution may be neutralized by admixing the neutralizing agent, either to a predetermined degree of preneutralization with subsequent post-neutralization to the final value after or during the polymerization reaction, or the monomer solution is directly adjusted to the final value by admixing the neutralizing agent before polymerization. The base polymer can be mechanically comminuted, for example by means of a meat grinder, in which case the neutralizing agent can be sprayed, sprinkled or poured on and then carefully mixed in. To this end, the gel mass obtained can be repeatedly minced for homogenization.

The neutralized base polymer is then dried with a belt, fluidized bed, tower dryer or drum dryer until the residual moisture content is preferably below 13% by weight, especially below 8% by weight and most preferably below 4% by weight, the water content being determined according to EDANA's recommended test method No. 430.2-02 “Moisture content” (EDANA=European Disposables and Nonwovens Association). The dried base polymer is thereafter ground and sieved, useful grinding apparatus typically include roll mills, pin mills, hammer mills, jet mills or swing mills.

The water-absorbing polymers to be used can be post-crosslinked in one version of the present invention. Useful post-crosslinkers v) include compounds comprising two or more groups capable of forming covalent bonds with the carboxylate groups of the polymers. Useful compounds include for example alkoxysilyl compounds, polyaziridines, polyamines, polyamidoamines, di- or polyglycidyl compounds as described in EP-A 083 022, EP-A 543 303 and EP-A 937 736, polyhydric alcohols as described in DE-C 33 14 019. Useful post-crosslinkers v) are further said to include by DE-A 40 20 780 cyclic carbonates, by DE-A 198 07 502 2-oxazolidone and its derivatives, such as N-(2-hydroxyethyl)-2-oxazolidone, by DE-A 198 07 992 bis- and poly-2-oxazolidones, by DE-A 198 54 573 2-oxotetrahydro-1,3-oxazine and its derivatives, by DE-A 198 54 574 N-acyl-2-oxazolidones, by DE-A 102 04 937 cyclic ureas, by DE-A 103 34 584 bicyclic amide acetals, by EP-A 1 199 327 oxetanes and cyclic ureas and by WO 03/031482 morpholine-2,3-dione and its derivatives.

Post-crosslinking is typically carried out by spraying a solution of the post-crosslinker onto the base polymer or the dry base-polymer particles. Spraying is followed by thermal drying, and the post-crosslinking reaction can take place not only before but also during drying.

Preferred post-crosslinkers v) are amide acetals or carbamic esters of the general formula I

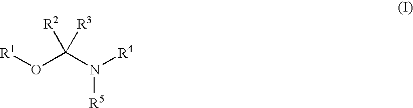

where
R¹ is C₁-C₁₂-alkyl, C₂-C₁₂-hydroxyalkyl, C₂-C₁₂-alkenyl or C₆-C₁₂-aryl,
R² is X or OR⁶
R³ is hydrogen, C₁-C₁₂-alkyl, C₂-C₁₂-hydroxyalkyl, C₂-C₁₂-alkenyl or C₆-C₁₂-aryl, or X,
R⁴ is C₁-C₁₂-alkyl, C₂-C₁₂-hydroxyalkyl, C₂-C₁₂-alkenyl or C₆-C₁₂-aryl
R⁵ is hydrogen, C₁-C₁₂-alkyl, C₂-C₁₂-hydroxyalkyl, C₂-C₁₂-alkenyl, C₁-C₁₂-acyl or C₆-C₁₂-aryl,
R⁶ is C₁-C₁₂-alkyl, C₂-C₁₂-hydroxyalkyl, C₂-C₁₂-alkenyl, C₁-C₁₂-acyl or C₆-C₁₂-aryl and
X is a carbonyl oxygen common to R² and R³,
   wherein R¹ and R⁴ and/or R⁵ and R⁶ can be a bridged C₂-C₆-alkanediyl and wherein the above mentioned radicals R¹ to R⁶ can still have in total one to two free valences and can be attached through these free valences to at least one suitable basic structure, for example 2-oxazolidones, such as 2-oxazolidone and N-hydroxyethyl-2-oxazolidone, N-hydroxypropyl-2-oxazolidone, N-methyl-2-oxazolidone, N-acyl-2-oxazolidones, such as N-acetyl-2-oxazolidone, 2-oxotetrahydro-1,3-oxazine, bicyclic amide acetals, such as 5-methyl-1-aza-4,6-dioxabicyclo[3.3.0]octane, 1-aza-4,6-dioxa-bicyclo[3.3.0]octane and 5-isopropyl-1-aza-4,6-dioxabicyclo[3.3.0]octane, bis-2-oxazolidones and poly-2-oxazolidones;
   or polyhydric alcohols, in which case the molecular weight of the polyhydric alcohol is preferably less than 100 g/mol, preferably less than 90 g/mol, more preferably less than 80 g/mol and most preferably less than 70 g/mol per hydroxyl group and the polyhydric alcohol has no vicinal, geminal, secondary or tertiary hydroxyl groups, and polyhydric alcohols are either diols of the general formula IIa

HO—R⁶—OH (IIa)

where R⁶ is either an unbranched dialkyl radical of the formula —(CH₂)ₘ—, where m is an integer from 2 to 20 and preferably from 3 to 12, and both the hydroxyl groups are terminal, or an unbranched, branched or cyclic dialkyl radical
or polyols of the general formula lib

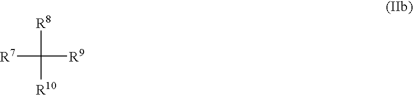

where R⁷, R⁸, R⁹ and R¹⁰ are independently hydrogen, hydroxyl, hydroxymethyl, hydroxyethyloxymethyl, 1-hydroxyprop-2-yloxymethyl, 2-hydroxypropyloxymethyl, methyl, ethyl, n-propyl, isopropyl, n-butyl, n-pentyl, n-hexyl, 1,2-dihydroxyethyl, 2-hydroxyethyl, 3-hydroxypropyl or 4-hydroxybutyl and in total 2, 3 or 4 and preferably 2 or 3 hydroxyl groups are present, and not more than one of R⁷, R⁸, R⁹ and R¹⁰ is hydroxyl, examples being ethyleneglycole, 1,3-propanediol, 1,4-butandiol, 1,5-pentanediol, 1,6-hexanediol and 1,7-heptanediol, 1,3-butanediol, 1,8-octanediol, 1,9-nonanediol and 1,10-decanediol, butane-1,2,3-triol, butane-1,2,4-triol, glycerol, trimethylolpropane, trimethylolethane, pentaerythritol, glycerol having 1 to 3 ethylene oxide units per molecule, trimethylolethane or trimethylolpropane each having 1 to 3 ethylene oxide units per molecule, propoxylated glycerol, trimethylolethane or trimethylolpropane each having 1 to 3 propylene oxide units per molecule, 2-tuply ethoxylated or propoxylated neopentylglycol,
or cyclic carbonates of the general formula III

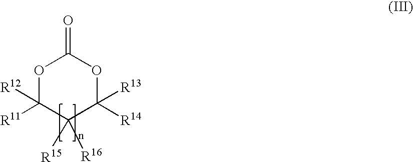

where R¹¹, R¹², R¹³, R¹⁴, R¹⁵ and R¹⁶ are independently hydrogen, methyl, ethyl, n-propyl, isopropyl, n-butyl, sec-butyl or isobutyl, and n is either 0 or 1, examples being ethylene carbonate and propylene carbonate,
or bisoxazolines of the general formula IV

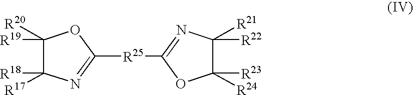

where R¹⁷, R¹⁸, R¹⁹, R²⁰, R²¹, R²², R²³ and R²⁴ are independently hydrogen, methyl, ethyl, n-propyl, isopropyl, n-butyl, sec-butyl or isobutyl and R²⁵ is a single bond, a linear, branched or cyclic C₁-C₁₂-dialkyl radical or polyalkoxydiyl radical which is constructed of one to ten ethylene oxide and/or propylene oxide units, and is comprised of polyglycol dicarboxylic acids for example. An example for a compound under formula IV being 2,2′-bis(2-oxazoline).

The at least one post-crosslinker v) is typically used in an amount of about 2.50 wt. % or less, preferably not more than 0.50% by weight, more preferably not more than 0.30% by weight and most preferably in the range from 0.001% and 0.15% by weight, all percentages being based on the base polymer, as an aqueous solution. It is possible to use a single post-crosslinker v) from the above selection or any desired mixtures of various post-crosslinkers.

The aqueous post-crosslinking solution, as well as the at least one post-crosslinker v), can typically further comprise a cosolvent. Cosolvents which are technically highly useful are C₁-C₆-alcohols, such as methanol, ethanol, n-propanol, isopropanol, n-butanol, sec-butanol, tert-butanol or 2-methyl-1-propanol, C₂-C₅-diols, such as ethylene glycol, 1,2-propylene glycol, 1,3-propanediol or 1,4-butanediol, ketones, such as acetone, or carboxylic esters, such as ethyl acetate.

A preferred embodiment does not utilize any cosolvent. The at least one post-crosslinker v) is then only employed as a solution in water, with or without an added deagglomerating aid. Deagglomerating aids are known to one skilled in the art and are described for example in DE-A 102 39 074 and also prior PCT application PCT/EP/05011073, which are each hereby expressly incorporated herein by reference. Preferred deagglomerating aids are surfactants such as ethoxylated and alkoxylated derivatives of 2-propylheptanol and also sorbitan monoesters. Particularly preferred deagglomerating aids are polyoxyethylene 20 sorbitan monolaurate and polyethylene glycol 400 monostearate.

The concentration of the at least one post-crosslinker v) in the aqueous post-crosslinking solution is for example in the range from 1% to 50% by weight, preferably in the range from 1.5% to 20% by weight and more preferably in the range from 2% to 10% by weight, based on the post-crosslinking solution.

In a further embodiment, the post-crosslinker is dissolved in at least one organic solvent and spray dispensed; in this case, the water content of the solution is less than 10 wt. %, preferably no water at all is utilized in the post-crosslinking solution.

It is however understood that post-crosslinkers which effect comparable surface-crosslinking results with respect to the final polymer performance may of course be used in this invention even when the water content of the solution containing such post-crosslinker and optionally a cosolvent is anywhere in the range of >0 to <100% by weight.

The total amount of post-crosslinking solution based on the base polymer is typically in the range from 0.3% to 15% by weight and preferably in the range from 2% to 6% by weight. The practice of post-crosslinking is common knowledge to those skilled in the art and described for example in DE-A 12 239 074 and also prior PCT application PCT/EP/05011073.

Spray nozzles useful for post-crosslinking are not subject to any restriction. Suitable nozzles and atomizing systems are described for example in the following literature references: Zerstäuben von Flüssigkeiten, Expert-Verlag, volume 660, Reihe Kontakt & Studium, Thomas Richter (2004) and also in Zerstäubungstechnik, Springer-Verlag, VDI-Reihe, Günter Wozniak (2002). Mono- and polydisperse spraying systems can be used. Suitable polydisperse systems include one-material pressure nozzles (forming a jet or lamellae), rotary atomizers, two-material atomizers, ultrasonic atomizers and impact nozzles. With regard to two-material atomizers, the mixing of the liquid phase with the gas phase can take place not only internally but also externally. The spray pattern produced by the nozzles is not critical and can assume any desired shape, for example a round jet, flat jet, wide angle round jet or circular ring. When two-material atomizers are used, the use of an inert gas will be advantageous. Such nozzles can be pressure fed with the liquid to be spray dispensed. The atomization of the liquid to be spray dispensed can in this case be effected by decompressing the liquid in the nozzle bore after the liquid has reached a certain minimum velocity. Also useful are one-material nozzles, for example slot nozzles or swirl or whirl chamber (full cone) nozzles (available for example from Düsen-Schlick GmbH, Germany or from Spraying Systems Deutschland GmbH, Germany). Such nozzles are also described in EP-A 0 534 228 and EP-A 1 191 051. In case that dispersions of insoluble inorganic salts or other fine insoluble particles are sprayed out of one solution with a post-cross-linker or out of a separate solution in parallel to the post-cross-linker solution, it is preferable to use two-material spray nozzles with external mixing chamber.

After spraying, the water-absorbing polymeric particles are thermally dried, and the post-crosslinking reaction can take place before, during or after drying.

The spraying with the solution of post-crosslinker is preferably carried out in mixers having moving mixing implements, such as screw mixers, paddle mixers, disk mixers, plowshare mixers and shovel mixers. Particular preference is given to vertical mixers and very particular preference to plowshare mixers and shovel mixers. Useful mixers include for example Lödige® mixers, Bepex® mixers, Nauta® mixers, Processall® mixers and Schugi® mixers.

Contact dryers are preferable, shovel dryers are more preferable and disk dryers are most preferable as the apparatus in which thermal drying is carried out. Suitable dryers include for example Bepex dryers and Nara® dryers. Fluidized bed dryers can be used as well, an example being Carman® dryers.

Drying can take place in the mixer itself, for example by heating the jacket or introducing a stream of warm inert gases. It is similarly possible to use a downstream dryer, for example a tray dryer, a rotary tube oven or a heatable screw. But it is also possible for example to utilize an azeotropic distillation as a drying process.

It is particularly preferable to apply the solution of post-crosslinker in a high speed mixer, for example of the Schugi-Flexomix® or Turbolizer® type, to the base polymer and the latter can then be thermally post-crosslinked in a reaction dryer, for example of the Nara-Paddle-Dryer® type or a disk dryer (i.e. Torus-Disc Dryer®, Hosokawa). The temperature of the base polymer can be in the range from 10 to 120° C. from preceding operations, and the post-crosslinking solution can have a temperature in the range from 0 to 150° C. More particularly, the post-crosslinking solution can be heated above room temperature to lower the viscosity. The preferred post-crosslinking and drying temperature range is from 30 to 220° C., especially from 120 to 210° C. and most preferably from 145 to 200° C. The preferred residence time at this temperature in the reaction mixer or dryer is preferably less than 100 minutes, more preferably less than 70 minutes and most preferably less than 40 minutes.

It is particularly preferable to utilize a fluidized bed dryer for the crosslinking reaction, and the residence time is then preferably below 30 minutes, more preferably below 20 minutes and most preferably below 10 minutes. In such fluidized bed dryer the post-crosslinking temperature is preferably in the range of 30 to 240° C., more preferably 120 to 220° C., and most preferably in the range 150 to 200° C.

The post-crosslinking dryer or fluidized bed dryer may be operated with air or dried air to remove vapors efficiently from the polymer.

The post-crosslinking dryer is preferably purged with an inert gas during the drying and post-crosslinking reaction in order that vapors may be removed and oxidizing gases, such as atmospheric oxygen, may be displaced. Mixtures of air and inert gases may also be used. To augment the drying process, the dryer and the attached assemblies are thermally well insulated and ideally fully heated. The inside of the post-crosslinking dryer is preferably at atmospheric pressure, or else at a slight under- or overpressure. The pressure inside may be kept constant or may be allowed to fluctuate. It is also possible to use pulsed air or pulsed inert gas in this process step.

To produce a very white polymer, the gas space in the dryer is kept as free as possible of oxidizing gases; at any rate, the volume fraction of oxygen in the gas space is not more than 14% by volume, preferably not more than 8% by volume, most preferably not more than 1% by volume.

The water-absorbing polymeric particles can have a particle size distribution in the range from 45 μm to 4000 μm. Particle sizes used in the hygiene sector preferably range from 45 μm to 1000 μm, preferably from 45-850 μm, and especially from 100 μm to 850 μm. It is preferable to coat water-absorbing polymeric particles having a narrow particle size distribution, especially 100-850 μm, or even 100-600 μm. A preferred narrow particle size distribution is obtained if the lower sifting screen for fines removal is selected from the range 100-300 μm (for example 150 μm or 200 μm), and the upper sifting screen for overs removal is selected from the range 600-1000 μm (for example 700 μm or 800 μm). The milling and sizing step in the process is typically a continuous operation. The extraction of the good product fraction which has the desired particle size is done by continuously removing the particles between the coarse and the fine-screen as described above. It is particularly useful to use at least one additional screen inbetween which retains a coarser part of the good product fraction and hereby avoids clogging of the fine screen in the bottom by reducing the load on this screen. Industrial useful screening methods and equipment is described in “Sieben und Siebmaschinen”, P. Schmidt, R. Körber, M. Coppers, Wiley V C H, 2003 which is expressly incorporated herein by reference.

Narrow particle size distributions are those in which not less than 80% by weight of the particles, preferably not less than 90% by weight of the particles and most preferably not less than 95% by weight of the particles are within the selected range; this fraction can be determined using the familiar sieve method of EDANA 420.2-02 “Particle Size Distribution”. Selectively, optical methods can be used as well, provided these are calibrated against the accepted sieve method of EDANA.

Preferred narrow particle size distributions have a span of not more than 700 μm, more preferably of not more than 600 μm, and most preferably of less than 400 μm. Span here refers to the difference between the coarse sieve and the fine sieve which bound the distribution. The coarse sieve is not coarser than 850 μm and the fine sieve is not finer than 45 μm. Particle size ranges which are preferred for the purposes of the present invention are for example fractions of 150-600 μm (span: 450 μm), of 100-700 μm, of 200-700 μm (span: 500 μm), of 150-700 μm, of 200-600 μm (span: 400 μm), of 200-800 μm (span: 600 μm), of 150-850 μm (span: 700 μm), of 300-700 μm (span: 400 μm), of 400-800 μm (span: 400 μm).

Preference is likewise given to monodisperse water-absorbing polymeric particles as obtained from the inverse suspension polymerization process. It is similarly possible to select mixtures of monodisperse particles of different diameter as water-absorbing polymeric particles, for example mixtures of monodisperse particles having a small diameter and monodisperse particles having a large diameter. It is similarly possible to use mixtures of monodisperse with polydisperse water-absorbing polymeric particles.

Coating these water-absorbing polymeric particles having narrow particle size distributions with a maximum particle size of 850 μm, more preferably having a maximum particle size of ≦700 μm, and most preferably having a maximum particle size of ≦600 μm according to the present invention in a continuous fluidized bed process provides an extremely homogeneously coated water-absorbing material, which has certain improved properties like better fluid permeability, improved anti-caking, or anti-microbial effects—depending on its particular surface coating—which are very consistent from lot to lot and therefore is particularly preferred.

The water-absorbing particles can be spherical in shape as well as irregularly shaped particles.

According to the invention the water-absorbing polymeric particles are spray-coated with a non-reactive coating agent. A non-reactive coating agent refers herein to a coating agent, which is substantially non-covalently bonded to the surface of the water-absorbing polymeric particles.

Preferred non-reactive coating agents are selected from the group consisting of water-insoluble inorganic powders, water-soluble multivalent metal salts, polycationic polymers and binding agents. Preference is given to water-insoluble inorganic powders and water-soluble multivalent metal salts.

According to the invention the polymeric particles are spray-coated with a non-reactive coating agent, which does not comprise an elastic film-forming polymer. The term “do not comprise” means that the elastic film-forming polymer is not present in an effective amount. The amount at which the elastomeric film-forming polymer will not affect the properties of the water-absorbing particles will be general less than 0.1% in particular less than 0.05% based on the weight of the water-absorbing polymeric particles. In particular the elastomeric film-forming polymer is completely absent. Film-forming means that the respective polymer can readily be made into a layer or coating upon evaporation of the solvent in which it is dissolved or dispersed. Elastomeric means the material will exhibit stress-induced deformation that is partially or completely reversed upon removal of the stress. Polymers having film-forming and also elastic properties include for example copolyesters, copolyamides, silicones, styrene-isoprene block copolymers, styrene-butadiene block copolymers and polyurethanes.

Suitable water-insoluble inorganic powders are for example water-insoluble salts, clays, limestone, talcum and zeolites. Such inorganic powders are described in WO 02/060983, which is hereby expressly incorporated herein by reference. A water-insoluble salt refers herein to a salt, which at a pH of 7 has a solubility in water of less than 5 g/l.

When a salt occurs in various crystal forms, all crystal forms of the salt shall be included. Suitable cations in the water-insoluble salt are for example Ca²⁺, Mg²⁺, Al³⁺, Sc³⁺, Y³⁺, Ln³⁺ (where Ln denotes lanthanoids), Ti⁴⁺, Zr⁴⁺, Li⁺, K⁺, Na⁺ or Zn²⁺. Suitable inorganic anionic counterions are for example carbonate, sulfate, bicarbonate, orthophosphate, silicate, oxide or hydroxide. Particularly preferred are water-insoluble salts like phosphates of Mg, Ca, Zn, Al, Cu, Fe and Ag.

The water-insoluble inorganic salts are preferably selected from calcium sulfate, calcium carbonate, calcium phosphate, calcium silicate, calcium fluoride, apatite, bor phosphate, aluminum phosphate, iron phosphate, cupper phosphate, silver phosphate, magnesium phosphate, magnesiumhydroxide, magnesium oxide, magnesium carbonate, dolomite, lithium carbonate, lithium phosphate, zinc oxide, zinc phosphate, oxides, hydroxides, carbonates and phosphates of the lanthanoids, sodium lanthanoid sulfate, scandium sulfate, yttrium sulfate, lanthanum sulfate, scandium hydroxide, scandium oxide, aluminum oxide, hydrated aluminum oxide and mixtures thereof. Apatite refers to fluoroapatite, hydroxyl apatite, chloroapatite, carbonate apatite and carbonate fluoroapatite. Of particular suitability are calcium and magnesium salts such as calcium carbonate, calcium phosphate, magnesium carbonate, calcium oxide, magnesium oxide, calcium sulfate and mixtures thereof. Amorphous or crystalline forms of aluminum oxide, titanium dioxide and silicon dioxide are also suitable. These non-reactive coating agents can also be used in their hydrated forms. Particularly preferred are the insoluble metal phosphates and inorganic compounds disclosed in U.S. Pat. No. 6,831,122 B2 which is expressly incorporated by reference herein.

Useful water-insoluble inorganic powders further include many clays, limestone, talcum and zeolites. Silicon dioxide is preferably used in its amorphous form, for example as hydrophilic or hydrophobic Aerosil®, as fumed silicas.

The average particle size of the finely divided water-insoluble inorganic powder is typically less than 200 μm, preferably less than 100 μm, especially less than 50 μm, more preferably less than 20 μm, even more preferably less than 10 μm and most preferably in the range of less than 5 μm. Fumed silicas are often used as even finer particles, e.g. less than 50 nm, preferably less than 30 nm, even more preferably less than 20 nm primary particle size. In a particular preferred embodiment the average particle size of the finely divided water-insoluble salt is between 2-20 μm, most preferably between 4-10 μm. Inorganic powders with a particle size between 10-100 μm or preferably 10-50 μm are also very suitable and are preferred in cases when the fine dust content below 10 μm has to be minimized.

In a preferred embodiment, the finely divided water-insoluble inorganic powder is used in an amount in the range from 0.001% to 20% by weight, preferably less than 10% by weight, especially in the range from 0.001% to 5% by weight, more preferably in the range from 0.001% to 2% by weight and most preferably in the range from 0.1 and 1% by weight, based on the weight of the water-absorbing polymeric particles.

A water-soluble salt refers herein to a salt, which at a pH of 7 has a solubility in water of ≧5 g/l. Suitable water-soluble multivalent metal salts are for example—but not limited to—Ca²⁺, Mg²⁺, Zn²⁺, Al³⁺, Fe^{2+/3+} which may be used as any of their sufficiently water soluble salts, with the sulfates being most preferred. Such multivalent metal salts are described in WO 05/080479, which is hereby expressly incorporated herein by reference. Other suitable water-soluble metal salts are commercially available aqueous silica sol, such as for example Levasil® Kiselsole (H. C. Starck GmbH), which have particle sizes in the range 5-75 nm.

In a preferred embodiment, the water-soluble salt is used in an amount in the range from 0.001% to 20% by weight, preferably less than 10% by weight, especially in the range from 0.001% to 5% by weight, more preferably in the range from 0.001% to 2% by weight and most preferably in the range from 0.1 and 1% by weight, based on the weight of the water-absorbing polymeric particles.

Suitable polycationic polymers in the present invention are for example—but not limited to—polyethyleneimine, polyallylamine, polyvinylamine, and partially hydrolyzed polyvinylformamide or poylvinylacetamide. Such polycationic polymers are described in WO 04/024816, which is hereby expressly incorporated herein by reference.

Suitable binding agents in the present invention are for example—but not limited to—dendritic and hyperbranched polymers, preferably hydrophilic dendritic or hyperbranched polymers like polyglycerine, or hydrophilic polymers like polyethylenglycole, polyvinylalcohole, polypropyleneglycole, polyvinylpyrrolidone. Preferable binding agents are the 1-100 tuply ethoxylated and/or propoxylated derivatives of tri- or polyfunctional polyols like glycerine, trimethylolpropane, trimethylolethane, pentaerythrit, sorbitol and the like. Polyvinylalcohole and polyvinylpyrrolidone are preferred in an amount <0.5% by weight, preferably <0.1% by weight, based on the weight of the water-absorbing polymeric particles. Further preferred are polyols with a molecular weight above 100 g/mole, polymers with a T_{g}<50° C. Particularly preferred binding agents from the group of polyols are triethanolamine, pentaerythrit, glycerine, 5- to 100-tuply ethxoylated glycerine, trimethylolpropane, trimethylolethane, pentaerytritol, dipentaerythritol, sorbitol, erythritol and the like. Other examples for binding agents are polyethylenoxides with a molecular weight of between 100 g/mole and 20000 g/mole.

Suitable other non-reactive coating agents are for example waxes, stearic acid and stearates, surfactants or preferably saw dust. Saw dust is preferably applied in combination with a binding agent.

Waxes and preferably micronized or preferably partially oxidized polyethylenic waxes, which can likewise be used in the form of an aqueous dispersion are described in EP 0 755 964, which is hereby expressly incorporated herein by reference. A wax is independent of its chemical composition herein defined according to the “Deutsche Gesellschaft für Fettwissenschaft (DGF)” from 1974 in “DGF-Einheitsmethoden: Untersuchung von Fetten, Fettprodukten und verwandten Stoffen, Abteilung M: Wachse und Wachsprodukte; Wissenschaftliche Verlagsgesellschaft, Stuttgart, 1975”.

Useful non-reactive coating agents further include stearic acid, stearates—for example: magnesium stearate, calcium stearate, zinc stearate, aluminum stearate, and furthermore polyoxyethylene-20-sorbitan monolaurate and also polyethylene glycol 400 monostearate.

Useful non-reactive coating agents likewise include surfactants. A surfactant can be used alone or mixed with one of the abovementioned non-reactive coating agents, preferably a water-insoluble salt.

Useful surfactants include nonionic, anionic and cationic surfactants and also mixtures thereof. The water-absorbing material preferably comprises nonionic surfactants. Useful nonionic surfactants include for example sorbitan esters, such as the mono-, di- or triesters of sorbitans with C₈-C₁₈-carboxylic acids such as lauric, palmitic, stearic and oleic acids; polysorbates; alkylpolyglucosides having 8 to 22 and preferably 10 to 18 carbon atoms in the alkyl chain and 1 to 20 and preferably 1.1 to 5 glucoside units; N-alkylglucamides; alkylamine alkoxylates or alkylamide ethoxylates; alkoxylated C₈-C₂₂-alcohols such as fatty alcohol alkoxylates or oxo alcohol alkoxylates; block polymers of ethylene oxide, propylene oxide and/or butylene oxide; alkylphenol ethoxylates having C₆-C₁₄-alkyl chains and 5 to 30 mol of ethylene oxide units.

The amount of surfactant is generally in the range from 0.001% to 0.5% by weight, preferably less than 0.1% by weight and especially below 0.05% by weight, based on the weight of the water-absorbing material.

Sawdust can exhibit good anti-microbial properties and may be used as it is or in an activated form as is described in EP 1 005 964. If sawdust is used as anti-microbial agent then many woods like larch, cedar, pine, or oak will show anti-microbial, anti-viral, or anti-fungicidal effects to some extent, woods like pine and oak are preferred. However, any wood that does show anti-microbial effects can be processed into sawdust and used as coating agent according to the present invention. The particle size of sawdust is typically less than 1000 μm, preferably less than 300 μm, and more preferably less than 100 μm. A particularly preferred saw dust exhibits anti-microbial properties and is useful for odor control superabsorbent polymers for incontinence products. The sawdust is applied in combination with a binding agent. In one particularly preferred embodiment of the present invention the coating with sawdust takes place under mild temperature conditions below 120° C.

Each non-reactive coating agent is used, if not mentioned otherwise, in an amount in the range from 0.001% to 20% by weight, preferably less than 10% by weight, especially in the range from 0.001% to 5% by weight, more preferably in the range from 0.001% to 2% by weight and most preferably in the range from 0.1 and 1% by weight, based on the weight of the water-absorbing polymeric particles.

Each non-reactive coating agent might be applied alone or in combination with another. In a particular preferred embodiment the water-insoluble inorganic powder is applied together with a binding agent, as hereinabove described, to affix the finely divided particles of the inorganic powder onto the water-absorbing polymeric particles, preferably simultaneous in the process. The fixation of the water-insoluble inorganic powder with such binding agent is particularly useful to avoid stripping of the water-insoluble inorganic powder from the particle surfaces by mechanical stress or air-flow during production or in processing of the water-absorbing material, or in use of the hygiene article.

The water-insoluble salts are used as a solid material or in the form of dispersions, preferably as an aqueous dispersion. Solids are typically jetted into the apparatus as fine dusts by means of a carrier gas. The dispersion is preferably applied by means of a high-speed stirrer by preparing the dispersion from solid material and water in a first step and introducing it in a second step rapidly into the fluidized bed preferably via a nozzle. The aqueous dispersion can if appropriate be applied together with another coating agent dispersed together or as a separate dispersion via separate nozzles at the same time as another coating agent or at different times from another coating agent. The insoluble inorganic salt and the binding agent can be sprayed most preferably out of one aqueous dispersion of the insoluble inorganic salt in which the binding agent is dissolved.

It is particularly preferable to apply the water-insoluble salt after a sticky coating agent has been applied or in parallel with such sticky coating agent or before such sticky coating agent is applied, and before the optional subsequent drying step. It is also possible to only coat the water-absorbing polymeric particles with such water-insoluble salt to impart very good anti-stick properties to the water-absorbing material under humid ambient conditions.

It is possible that the water-absorbing material comprises two or more layers of coating agent (shells), obtainable by coating the water-absorbing polymeric particles twice or more. This may be the same coating agent or a different coating agent. Particularly preferred coating agents are calciumphosphate together with a binding agent like 7-tuply ethoxylated trimethylolpropane or 7-tuply ethoxylated glycerol or with glycerol or with a polycationic polymer. Other preferred coating agents are aluminumsulfate combined with a surfactant or a polycationic polymer.

According to the present invention the particles are spray-coated in a continuous fluidized bed reactor. The water-absorbing particles are introduced as generally customary, depending on the type of the reactor, and are generally coated by spraying with the coating agent as an aqueous dispersion and/or aqueous solution. Aqueous dispersions of the inorganic powder which also contain a binding agent are particularly preferred. Most preferred binding agents are the ethoxylated polyols and glycerol as described hereinbefore.

The aqueous dispersion applied by spray-coating is preferably very concentrated. For this, the viscosity of this aqueous inorganic powder dispersion should not be too high, or the dispersion can no longer be finely dispersed for spraying. It is particularly preferred that the dispersion exhibits Newtonian flow or thixotropic flow.

The concentration of water-insoluble inorganic salt in the aqueous dispersion is generally in the range from 1% to 60% by weight, preferably in the range from 5% to 40% by weight and especially in the range from 10% to 30% by weight. Higher dilutions are possible, but generally lead to longer coating times.

Fluidized bed means that the polymeric particles are carried upwards by a gas stream which dilutes the phase of the solid particles, keeps the particles agitated, and balances gravity. Continuous fluidized bed means a reactor operating according to the foregoing principle in which continuously uncoated solid particles are fed into the reactor and after passing the reactor are continuously taken from the reactor. Typically the fluidized particles pass at least one spray zone or at least one spray chamber inside the reactor and may be coated by means of spraying a coating solution or dispersion out of nozzles into the fluidized bed of particles as described below.

Useful fluidized bed reactors include for example the fluidized or suspended bed coaters familiar in the pharmaceutical industry. Particular preference is given to reactors using the Wurster principles or the Glatt-Zeller principles which are described for example in “Pharmazeutische Technologie, Georg Thieme Verlag, 2nd edition (1989), pages 412-413” and also in “Arzneiformenlehre, Wissenschaftliche Verlagsbuchandlung mbH, Stuttgart 1985, pages 130-132”. Particularly suitable continuous fluidized bed processes on a commercial scale are described in Drying Technology, 20(2), 419-447 (2002).

According to a Wurster process the water-absorbing polymeric particles are carried by an upwardly directed stream of carrier gas in a central tube, against the force of gravity, past at least one spray nozzle and are sprayed concurrently with the finely disperse polymeric solution or dispersion. The particles thereafter fall back to the base along the side walls, are collected on the base, and are again carried by the flow of carrier gas through the central tube past the spray nozzle. The spray nozzle typically sprays from the bottom into the fluidized bed, it can also project from the bottom into the fluidized bed.

According to a Glatt-Zeller process, the water-absorbing polymeric particles are conveyed by the carrier gas on the outside along the walls in the upward direction and then fall in the middle onto a central nozzle head, which typically comprises at least 3 two-material nozzles, which spray to the side. The particles are thus sprayed from the side, fall past the nozzle head to the base and are taken up again there by the carrier gas, so that the cycle can start anew.

The feature common to the two processes is that the particles are repeatedly carried in the form of a fluidized bed past the spray device, whereby a very thin and typically very homogeneous shell can be applied. Furthermore, a carrier gas is used at all times and it has to be fed and moved at a sufficiently high rate to maintain fluidization of the particles. As a result, liquids are rapidly vaporized in the apparatus, such as for example the solvent (i.e. water) of the dispersion, even at low temperatures, whereby the coating agent particles of the dispersion are precipitated onto the surface of the particles of the water-absorbing polymer, which are to be coated. Useful carrier gases include the inert gases mentioned above and air or dried air or mixtures of any of these gases.

Suitable fluidized bed reactors work according to the principle that the coating agent solution or coating agent dispersion is finely atomized and the droplets randomly collide with the water-absorbing polymer particles in a fluidized bed, whereby a substantially homogeneous shell builds up gradually and uniformly after many collisions. The size of the droplets must be inferior to the particle size of the absorbent polymer. Droplet size is determined by the type of nozzle, the spraying conditions i.e. temperature, concentration, viscosity, pressure and typical droplets sizes are in the range 1 μm to 400 μm. A polymer particle size vs. droplet size ratio of at least 10 is typically observed. Small droplets with a narrow size distribution are favourable. The droplets of the atomized dispersion or solution are introduced either concurrently with the particle flow or from the side into the particle flow, and may also be sprayed from the top onto a fluidized bed. In this sense, other apparatus and equipment modifications which comply with this principle and which are likewise capable of building up fluidized beds are perfectly suitable for producing such effects.

Other continuous mixers not according to this invention and not using the fluidized bed principle like for example spray-mixers of the Telschig-type, Lödige Plow-share or Ruberg-mixers are not yielding a sufficiently homogeneous coating.

According to the present invention a continuous fluidized bed process is used and the spray is operated in top-, side- and/or bottom-mode. In a particularly preferred embodiment the spray is operated in side- and/or bottom-mode. A suitable apparatus is for example described in U.S. Pat. No. 5,211,985. Suitable apparatus are available also for example from Glatt Maschinen- und Apparatebau AG (Switzerland) as series GF (continuous fluidized bed) and as ProCell® spouted bed. The spouted bed technology uses a simple slot instead of a screen bottom to generate the fluidized bed and is particularly suitable for materials, which are difficult to fluidize.

Continuous multi-chamber or multi-zone processes are particularly preferred as they allow blending, dedusting and functional coating of water-absorbing polymeric particles with one or more sprayable components in one process step.

In other embodiments it may also be desired to operate the spray top- and bottom-mode, or it may be desired to spray from the side or from a combination of several different spray positions.

The process of the present invention utilizes the aforementioned nozzles, which are customarily used for post-crosslinking. However, two-material nozzles are particularly preferred and atomization is particularly effected by an inert gas.

It is advantageous that the fluidized bed gas stream, which enters from below is likewise chosen such that the total amount of the water-absorbing polymeric particles is fluidized in the apparatus. The gas velocity for the fluidized bed is above the minimum fluidization velocity (measurement method described in Kunii and Levenspiel “Fluidization engineering” 1991) and below the terminal velocity of water-absorbing polymer particles, preferably 10% above the minimum fluidization velocity. The gas velocity for the Wurster tube is above the terminal velocity of water-absorbing polymer particles, usually below 100 m/s, preferably 10% above the terminal velocity.

The gas stream acts to vaporize the water, or the solvents. In a preferred embodiment, the coating conditions of gas stream and temperature are chosen so that the relative humidity or vapor saturation at the exit of the gas stream is in the range from 0.10% to 90%, preferably from 1.0% to 80%, or preferably from 10% to 70% and especially from 30% to 60%, based on the equivalent absolute humidity prevailing in the carrier gas at the same temperature or, if appropriate, the absolute saturation vapor pressure.

The fluidized bed reactor may be built from stainless steel or any other typical material used for such reactors, also the product contacting parts may be stainless steel to accommodate the use of organic solvents and high temperatures.

In a further preferred embodiment, the inner surfaces of the fluidized bed reactor are at least partially coated with a material whose contact angle with water is more than 90° at 25° C. Teflon or polypropylene are examples of such a material. Preferably, all product-contacting parts of the apparatus are coated with this material.

The choice of material for the product-contacting parts of the apparatus, however, also depends on whether these materials exhibit strong adhesion to the utilized coating agent dispersion or solution or to the water-absorbing polymeric particles to be coated. Preference is given to selecting materials which have no such adhesion either to the polymeric particles to be coated or to the coating dispersion or solution in order that caking may be avoided.

According to the present invention, coating takes place at a product and/or carrier gas temperature in the range from 0° C. to 150° C., preferably from 0° C. to 120° C., preferably from 15 to 100° C., especially from 20 to 90° C. and most preferably from 20 to 70° C.

According to a particularly preferred embodiment of the present invention, coating takes place with water-absorbing polymeric particles which exhibit a temperature of at least 15° C., preferably at least 35° C., most preferably at least 60° C., and the carrier gas exhibits a temperature in the range from 0° C. to 120° C., preferably from 20° C. to 100° C., and the coating dispersion or solution exhibits a temperature from 0° C. to 100° C., preferably from 10° C. to 95° C., and most preferably from 20° C. to 45° C. before spraying.

In one preferred embodiment of the present invention the above coating is applied as an aqueous dispersion or solution in spray form in a continuous fluidized bed process without any heat treatment after the coating and also under mild temperature conditions during the coating, preferably at a product temperature of less than 120° C., more preferred at a product temperature of less than 70° C., and most preferred at a product temperature of less than 50° C.

In another preferred embodiment of the present invention the product is still held at elevated temperature of 50-140° C. for about 1-30 minutes after the coating step in the continuous fluidized bed itself or in an additional dryer which is passed by the product subsequent to coating.

According to the invention, drying optionally takes place at temperatures above 50° C.

The optional drying is carried out for example in a downstream fluidized bed dryer, a tunnel dryer, a tray dryer, a tower dryer, one or more heated screws or a disk dryer or a Nara® dryer. Drying is preferably done in a fluidized bed reactor and more preferably directly in the same continuous fluidized bed reactor used for coating.

The optional drying can take place on trays in forced air ovens.

In one embodiment for the process steps of coating, drying, and subsequent cooling, it may be possible to use ambient air or dried air in each of these steps. It is also possible and sometimes necessary to use air with a pre-set humidity level.

In other embodiments an inert gas may be used in one or more of these process steps. In yet another embodiment one can use mixtures of air and inert gas in one or more of these process steps.

It is very particularly preferable when the concluding cooling phase is carried out under protective gas too. Preference is therefore given to a process where the production of the water-absorbing material according to the present invention takes place under inert gas.

It is believed without wishing to be bound by theory that the water-absorbing material obtained by the process according to the present invention is surrounded by a very homogeneous distribution of finely divided particles or spots on each polymeric particle surface. It is furthermore believed without wishing to be bound by theory that the superior homogeneity of such distribution is important to impart very consistent physical use properties to the water-absorbing material.

After the optional drying step has been concluded, the dried water-absorbing materials are cooled. To this end, the warm and dry polymer is preferably continuously transferred into a downstream cooler. This can be for example a disk cooler, a Nara paddle cooler or a screw cooler. Cooling is via the walls and if appropriate the stirring elements of the cooler, through which a suitable cooling medium such as for example warm or cold water flows. Water may preferably be sprayed on in the cooler; this increases the efficiency of cooling (partial evaporation of water) and the residual moisture content in the finished product can be adjusted to a value in the range from 0% to 15% by weight, preferably in the range from 0.01% to 6% by weight and more preferably in the range from 0.1% to 3% by weight. The increased residual moisture content reduces the dust content of the product.

Optionally, however, it is possible to use the cooler for cooling only and to carry out the addition of water and additives in a downstream separate mixer. Cooling lowers the product temperature only to such an extent that the product can easily be packed in plastic bags or within silo trucks. Product temperature after cooling is typically less than 90° C., preferably less than 60° C., most preferably less than 40° C. and preferably more than −20° C.

Optionally there is a finished product screen after the continuous fluidized bed coater or after the optional heat treatment step or after the cooling step so that agglomerates formed during the process can be removed from the product.

It may be preferable to use a fluidized bed cooler. If coating and drying are both carried out in fluidized beds, the two operations can be carried out either in separate apparatuses or in one apparatus having communicating chambers. If cooling too is to be carried out in a fluidized bed cooler, it can be carried out in a separate apparatus or optionally combined with the other two steps in just one apparatus having a third reaction chamber. More reaction chambers are possible as it may be desired to carry out certain steps like the coating step in multiple chambers consecutively linked to each other, so that the water absorbing polymer particles consecutively build the coating shell in each chamber by successively passing the particles through each chamber one after another.

According to a preferred embodiment further water-absorbing polymeric particles are blended to the water-absorbing polymeric particles during or preferable before the coating step, the main stream water-absorbing polymeric particles preferably being surface-cross linked. Preferred polymeric particles for admixing are other grades and types of water-absorbing polymeric particles or off-spec materials for rework from the main stream polymer production process itself.

According to another embodiment useful components for admixing before or during the coating step are anti-microbial and/or odor control agents.

According to a preferred embodiment dedusting is achieved via gas-flow, preferably via air-flow, from this main stream water-absorbing polymeric particles and optionally from the components admixed to it. The water-absorbing polymeric particles are preferably surface-cross linked.

Preferred is a process for producing water-absorbing material, which comprises the steps of
a) spray-coating water-absorbing polymeric particles with an aqueous dispersion of an water-insoluble inorganic powder in a continuous process in a fluidized bed reactor, preferably, in the range from 0° C. to 120° C., and
b) drying the coated particles at a temperature above 50° C.

According to a preferred process post-crosslinked water absorbing polymeric particles A (main stream) are fed into a continuous fluidized bed reactor optional together with water absorbing polymeric particles different to the particles A and spray-coating the polymeric particles on their way through the reactor.

In one embodiment the particles subsequently pass through different zones A, B, C of the reactor one after another, where the coating agent, preferably different coating agents, are sprayed on the particle surface. The reactor comprises at least one zone and may comprise as many zones as needed to spray on the desired number and amount of coating agents, to accomplish blending with other granular particles that are to be mixed into the water-absorbing polymeric particles, and to accomplish dedusting of the water-absorbing polymeric particles or the water-absorbing material.

### Preferrably

a) a water-insoluble inorganic powder and/or a water soluble inorganic salt,
b) a binding agent and
c) optionally an odor control and/or anti-microbial agent
   are subsequently sprayed in this order.

In another embodiments the particles are subsequently spray-coated with a), c) and b) in this respective order.

In another embodiments the particles are subsequently spray-coated with b), a) and c) in this respective order.

In another embodiments the particles are subsequently spray-coated with b), c) and a) in this respective order.

In another embodiments the particles are subsequently spray-coated with c), a) and b) in this respective order.

In another embodiments the particles are subsequently spray-coated with c), b) and a), in this respective order.

A surfactant may be added to any of the foregoing spray solutions or may be sprayed on separately at any step in the process.

In another embodiment the particles are first dedusted by stripping of fine dust via the gas-stream in the front zones of the reactor and spray-coated in succession with at least two coating agents, for example with b) and a) in this order, or preferably with a) and b) in this order.

In yet another embodiment the particles are first dedusted by stripping of fine dust via the gas-stream in the front zone of the reactor and spray-coated in succession with at least two coating agents, preferably with b) and a) and b) in this order.

In these embodiments a surfactant may be added to any of the foregoing spray solutions or may be sprayed on separately at any step in the process.

Preference is given to a process comprising the steps of
a) feeding post-crosslinked water absorbing polymeric particles A (main stream) into a continuous fluidized bed reactor optionally together with water absorbing polymeric particles different to the particles A,
b) dedusting the water-absorbent polymeric particles by stripping off any particles less than 10 μm by a gas stream,
c) spray-coating the water-absorbing polymeric particles with a dispersion and/or a solution of a non reactive coating agent preferably at temperatures in the range from 0° C. to 120° C. preferably in the range from 10° C. to 90° C.,
d) optionally drying the coated particles at a temperature above 50° C. and subsequently
e) cooling the dried particles to a temperature below 90° C.

The present invention relates further to the water-absorbing material received according to the process described above. It relates further to the water-absorbing material received according to the inventive process comprising the step of spray-coating water-absorbing polymeric particles with sawdust and optionally a binding agent. In one embodiment the water-absorbing polymeric particles are jet-coated with sawdust and spray-coated with a binding agent.

The present invention provides water-absorbing material having a high centrifuge retention capacity (CRC), high absorbency under load (AUL) and high saline flow conductivity (SFC), the water-absorbing material having to have high and consistent saline flow conductivity (SFC) in particular.

The present invention provides further a process that allows a continuous and homogeneous coating of water-insoluble inorganic powders onto the surfaces of the water-absorbing polymeric particles preferably in combination with a binding agent, to the water-absorbing polymeric particle surfaces. Useful classes of fine particles for coating are particles which increase the CRC vs. SFC-balance, or which provide anti-caking properties, or which improve odor control, or which impart anti-microbial properties to the granular main stream water-absorbing polymeric particles. By coating with binding agents it is also desired to provide another means for efficient dedusting in this process step. The main stream water-absorbing polymeric particles are preferably surface-cross linked.

The present invention provides a process that allows a continuous and homogeneous coating of multi-valent metal salts or polycationic polymers onto the surfaces of the main stream water-absorbing polymeric particles by spraying them on from aqueous solution to the particle surfaces so that a very homogeneous coating results—as can be established by electron microscopy. The main stream water-absorbing polymeric particles is preferably surface-cross linked.

In a particularly preferred process the blending, dedusting, and coating steps are processed in one and the same process step with the main stream water-absorbing polymeric particles being surface-cross linked.

The coated water-absorbing polymeric particles may be present in the water-absorbing material of the invention mixed with other components, such as fibers, (fibrous) glues, organic or inorganic filler materials or flowing aids, process aids, anti-caking agents, odor control agents, coloring agents, coatings to impart wet stickiness, hydrophilic surface coatings, etc.

The water-absorbing material is typically obtainable by the process described herein, which is such that the resulting material is solid; this includes gels, flakes, fibers, agglomerates, large blocks, granules, particles, spheres and other forms known in the art for the water-absorbing polymeric particles described hereinafter.

The water-absorbing material of the invention preferably comprises less than 20% by weight of water, or even less than 10% or even less than 8% or even less than 5%, or even no water. The water content of the water-absorbing material can be determined by the Edana test, number ERT 430.1-99 (February 1999) which involves drying the water-absorbing material at 105° Celsius for 3 hours and determining the moisture content by the weight loss of the water-absorbing materials after drying.

The coating process of the present invention is notable for the fact that even difficult to apply coating agents result in a homogeneous coating. It is further possible to apply thermal sensitive coatings.

The resulting water absorbing materials show an unusual beneficial and consistent combination of absorbent capacity as measured in the CRC test and permeability as measured in the SFC test described herein, and moreover the resulting water absorbing materials show very low within-lot and from-lot-to-lot variation. A lot is defined as the quantity of product produced from a continuous production process in a defined time period—for example within 24 hours. Several samples may be taken from one or from different lots and may be analysed for product performance.

Preference is given to a water-absorbing material whose Centrifuge Retention Capacity (CRC) value is not less than 20 g/g, preferably not less than 25 g/g.

Preference is likewise given to a water-absorbing material where the SFC (Saline Flow Capacity) is at least 50×10⁻⁷ cm³s/g, preferably at least 90×10⁻⁷ cm³s/g and where the CRC is not less than 27 g/g, preferably not less than 28 g/g, more preferably not less than 29 g/g, most preferably at least 30 g/g.

The present invention is useful as it allows the easy modification of the properties of a water-absorbing polymeric particles after its production and hereby allows high flexibility in the product grades generated from a production plant although the base polymer production and the surface-cross-linking step may be run with constant recipe and process conditions in the production step to optimize throughput in these steps. The use of continuous fluidized bed reactors is particularly preferred in order to keep the production cost low. The current continuous process is very economic since the operation of these processes as batch-process requires numbering-up to obtain useful throughputs for the modification of water-absorbing polymeric particles.

The process of the present invention is notable for the fact that it produces water-absorbing polymeric material with excellent absorbing properties in a good time-space yield.

The water-absorbing material is useful in hygiene articles as baby diapers or incontinence products and packaging material.

The water-absorbing material, hereinafter also referred to as hydrogel-forming polymer, was tested by the test methods described hereinbelow.

### Methods:

The measurements should be carried out, unless otherwise stated, at an ambient temperature of 23±2° C. and a relative humidity of 50±10%. The water-absorbing polymeric particles are thoroughly mixed through before measurement. For the purpose of the following methods AGM means “Absorbent Gelling Material” and can relate to the water absorbing polymer particles as well as to the water-absorbing material. The respective meaning is clearly defined by the data given in the examples below.

### CRC (Centrifuge Retention Capacity)

This method determines the free swellability of the hydrogel in a teabag. To determine CRC, 0.2000+/−0.0050 g of dried hydrogel (particle size fraction 106-850 μm or as specifically indicated in the examples which follow) is weighed into a teabag 60×85 mm in size, which is subsequently sealed shut. The teabag is placed for 30 minutes in an excess of 0.9% by weight sodium chloride solution (at least 0.83 l of sodium chloride solution/1 g of polymer powder). The teabag is subsequently centrifuged at 250 g for 3 minutes. The amount of liquid is determined by weighing the centrifuged teabag. The procedure corresponds to that of EDANA recommended test method No. 441.2-02 (EDANA=European Disposables and Nonwovens Association). The teabag material and also the centrifuge and the evaluation are likewise defined therein.

AUL (Absorbency Under Load 0.7 psi)

Absorbency Under Load is determined similarly to the absorption under pressure test method No. 442.2-02 recommended by EDANA (European Disposables and Nonwovens Association), except that for each example the actual sample having the particle size distribution reported in the example is measured.

The measuring cell for determining AUL 0.7 psi is a Plexiglas cylinder 60 mm in internal diameter and 50 mm in height. Adhesively attached to its underside is a stainless steel sieve bottom having a mesh size of 36 μm. The measuring cell further includes a plastic plate having a diameter of 59 mm and a weight which can be placed in the measuring cell together with the plastic plate. The weight of the plastic plate and the weight together weigh 1345 g. AUL 0.7 psi is determined by determining the weight of the empty Plexiglas cylinder and of the plastic plate and recording it as W₀. Then 0.900+/−0.005 g of hydrogel-forming polymer (particle size distribution 150-800 μm or as specifically reported in the examples which follow) is weighed into the Plexiglas cylinder and distributed very uniformly over the stainless steel sieve bottom. The plastic plate is then carefully placed in the Plexiglas cylinder, the entire unit is weighed and the weight is recorded as Wₐ. The weight is then placed on the plastic plate in the Plexiglas cylinder. A ceramic filter plate 120 mm in diameter, 10 mm in height and 0 in porosity (Duran, from Schott) is then placed in the middle of the Petri dish 200 mm in diameter and 30 mm in height and sufficient 0.9% by weight sodium chloride solution is introduced for the surface of the liquid to be level with the filter plate surface without the surface of the filter plate being wetted. A round filter paper 90 mm in diameter and <20 μm in pore size (S&S 589 Schwarzband from Schleicher & Schüll) is subsequently placed on the ceramic plate. The Plexiglas cylinder holding hydrogel-forming polymer is then placed with the plastic plate and weight on top of the filter paper and left there for 60 minutes. At the end of this period, the complete unit is taken out of the Petri dish from the filter paper and then the weight is removed from the Plexiglas cylinder. The Plexiglas cylinder holding swollen hydrogel is weighed out together with the plastic plate and the weight is recorded as W_{b}.

Absorbency under load (AUL) is calculated as follows:

AUL 0.7 psi [g/g]=[W_{b}−Wₐ]/[Wₐ−W₀]

AUL 0.3 psi and 0.5 psi are measured similarly at the appropriate lower pressure.

### Saline Flow Conductivity (SFC)

The method to determine the permeability of a swollen gel layer is the “Saline Flow Conductivity” also known as “Gel Layer Permeability” and is described in EP A 640 330.

The equipment used for this method has been modified as described below.

FIG. 1 shows the permeability measurement equipment set-up with the open-ended tube for air admittance A, stoppered vent for refilling B, constant hydrostatic head reservoir C, Lab Jack D, delivery tube E, stopcock F, ring stand support G, receiving vessel H, balance I and the SFC apparatus L.

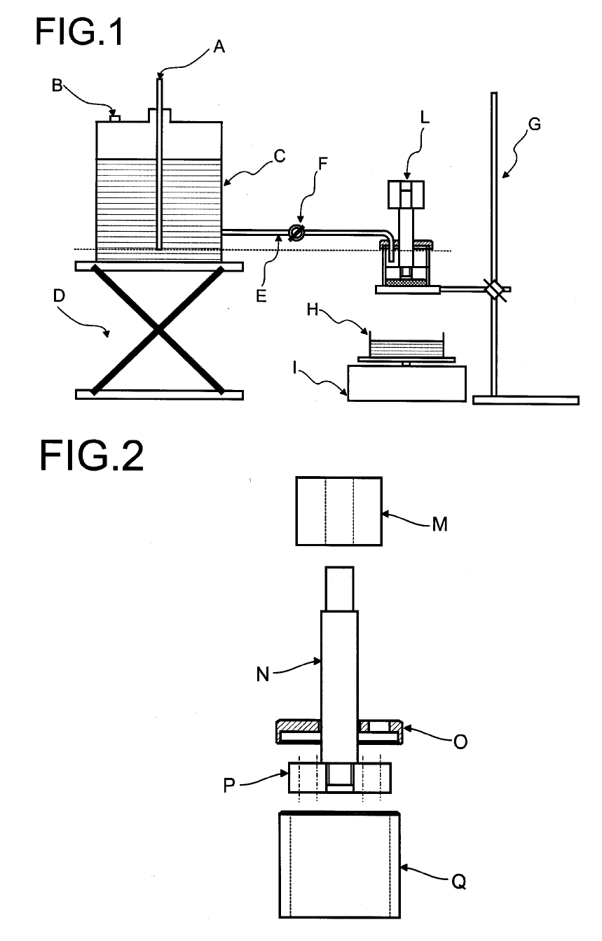

FIG. 2 shows the SFC apparatus L consisting of the metal weight M, the plunger shaft N, the lid 0, the center plunger P und the cylinder Q.

The cylinder Q has an inner diameter of 6.00 cm (area=28.27 cm²). The bottom of the cylinder Q is faced with a stainless-steel screen cloth (mesh width: 0.036 mm; wire diameter: 0.028 mm) that is bi-axially stretched to tautness prior to attachment. The plunger consists of a plunger shaft N of 21.15 mm diameter. The upper 26.0 mm having a diameter of 15.8 mm, forming a collar, a perforated center plunger P which is also screened with a stretched stainless-steel screen (mesh width: 0.036 mm; wire diameter: 0.028 mm), and annular stainless steel weights M. The annular stainless steel weights M have a center bore so they can slip on to plunger shaft and rest on the collar. The combined weight of the center plunger P, shaft and stainless-steel weights M must be 596 g (±6 g), which corresponds to 0.30 PSI over the area of the cylinder. The cylinder lid O has an opening in the center for vertically aligning the plunger shaft N and a second opening near the edge for introducing fluid from the reservoir into the cylinder Q.

The cylinder Q specification details are:

Outer diameter of the Cylinder: 70.35 mm
Inner diameter of the Cylinder: 60.0 mm

### Height of the Cylinder: 60.5 mm

The cylinder lid 0 specification details are:

Outer diameter of SFC Lid: 76.05 mm
Inner diameter of SFC Lid: 70.5 mm
Total outer height of SFC Lid: 12.7 mm
Height of SFC Lid without collar: 6.35 mm
Diameter of hole for Plunger shaft positioned in the center: 22.25 mm
Diameter of hole in SFC lid: 12.7 mm
Distance centers of above mentioned two holes: 23.5 mm

The metal weight M specification details are:

Diameter of Plunger shaft for metal weight: 16.0 mm
Diameter of metal weight: 50.0 mm
Height of metal weight: 39.0 mm

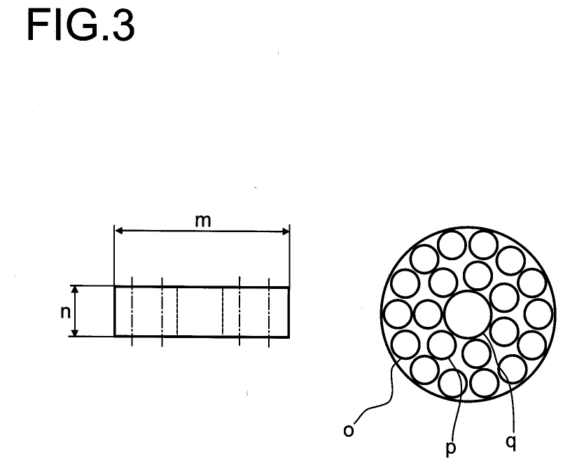

FIG. 3 shows the plunger center P specification details

Diameter m of SFC Plunger center: 59.7 mm
Height n of SFC Plunger center: 16.5 mm
14 holes o with 9.65 mm diameter equally spaced on a 47.8 mm bolt circle and 7 holes p with a diameter of 9.65 mm equally spaced on a 26.7 mm bolt circle ⅝ inches thread q

Prior to use, the stainless steel screens of SFC apparatus, should be accurately inspected for clogging, holes or over stretching and replaced when necessary. An SFC apparatus with damaged screen can deliver erroneous SFC results, and must not be used until the screen has been fully replaced.

Measure and clearly mark, with a permanent fine marker, the cylinder at a height of 5.00 cm (±0.05 cm) above the screen attached to the bottom of the cylinder. This marks the fluid level to be maintained during the analysis. Maintenance of correct and constant fluid level (hydrostatic pressure) is critical for measurement accuracy.

A constant hydrostatic head reservoir C is used to deliver NaCl solution to the cylinder and maintain the level of solution at a height of 5.0 cm above the screen attached to the bottom of the cylinder. The bottom end of the reservoir air-intake tube A is positioned so as to maintain the fluid level in the cylinder at the required 5.0 cm height during the measurement, i.e., the height of the bottom of the air tube A from the bench top is the same as the height from the bench top of the 5.0 cm mark on the cylinder as it sits on the support screen above the receiving vessel. Proper height alignment of the air intake tube A and the 5.0 cm fluid height mark on the cylinder is critical to the analysis. A suitable reservoir consists of a jar containing: a horizontally oriented L-shaped delivery tube E for fluid delivering, an open-ended vertical tube A for admitting air at a fixed height within the reservoir, and a stoppered vent B for re-filling the reservoir. The delivery tube E, positioned near the bottom of the reservoir C, contains a stopcock F for starting/stopping the delivery of fluid. The outlet of the tube is dimensioned to be inserted through the opening in the cylinder lid O, with its end positioned below the surface of the fluid in the cylinder (after the 5 cm height is attained). The air-intake tube is held in place with an o-ring collar. The reservoir can be positioned on a laboratory jack D in order to adjust its height relative to that of the cylinder. The components of the reservoir are sized so as to rapidly fill the cylinder to the required height (i.e., hydrostatic head) and maintain this height for the duration of the measurement. The reservoir must be capable to deliver liquid at a flow rate of minimum 3 g/sec for at least 10 minutes.

Position the plunger/cylinder apparatus on a ring stand with a 16 mesh rigid stainless steel support screen (or equivalent). This support screen is sufficiently permeable so as to not impede fluid flow and rigid enough to support the stainless steel mesh cloth preventing stretching. The support screen should be flat and level to avoid tilting the cylinder apparatus during the test. Collect the fluid passing through the screen in a collection reservoir, positioned below (but not supporting) the support screen. The collection reservoir is positioned on a balance accurate to at least 0.01 g. The digital output of the balance is connected to a computerized data acquisition system.

### Preparation of Reagents

Following preparations are referred to a standard 1 liter volume. For preparation multiple than 1 liter, all the ingredients must be calculated as appropriate.

### Jayco Synthetic Urine

Fill a 1 L volumetric flask with de-ionized water to 80% of its volume, add a stir bar and put it on a stirring plate. Separately, using a weighing paper or beaker weigh (accurate to ±0.01 g) the amounts of the following dry ingredients using the analytical balance and add them into the volumetric flask in the same order as listed below. Mix until all the solids are dissolved then remove the stir bar and dilute to 1 L volume with distilled water. Add a stir bar again and mix on a stirring plate for a few minutes more. The conductivity of the prepared solution must be 7.6±0.23 mS/cm.

Chemical Formula Anhydrous Hydrated

### Potassium Chloride (KCl) 2.00 g

### Sodium Sulfate (Na₂SO₄) 2.00 g

Ammonium dihydrogen phosphate (NH₄H₂PO₄) 0.85 g
Ammonium phosphate, dibasic ((NH₄)₂HPO₄) 0.15 g

### Calcium Chloride (CaCl₂) 0.19 g (2H₂O) 0.25 g

Magnesium chloride (MgCl₂) 0.23 g (6H₂O) 0.50 g

To make the preparation faster, wait until total dissolution of each salt before adding the next one. Jayco may be stored in a clean glass container for 2 weeks. Do not use if solution becomes cloudy. Shelf life in a clean plastic container is 10 days.

### 0.118 M Sodium Chloride (NaCl) Solution

Using a weighing paper or beaker weigh (accurate to ±0.01 g) 6.90 g of sodium chloride into a 1 L volumetric flask and fill to volume with de-ionized water. Add a stir bar and mix on a stirring plate until all the solids are dissolved. The conductivity of the prepared solution must be 12.50±0.38 mS/cm.

### Test Preparation

Using a reference metal cylinder (40 mm diameter; 140 mm height) set the caliper gauge (e.g. Mitotoyo Digimatic Height Gage) to read zero. This operation is conveniently performed on a smooth and level bench top. Position the SFC apparatus without AGM under the caliper gauge and record the caliper as L1 to the nearest of 0.01 mm.

Fill the constant hydrostatic head reservoir with the 0.118 M NaCl solution. Position the bottom of the reservoir air-intake tube A so as to maintain the top part of the liquid meniscus in the SFC cylinder at the required 5.0 cm height during the measurement. Proper height alignment of the air-intake tube A at the 5 cm fluid height mark on the cylinder is critical to the analysis.

Saturate an 8 cm fritted disc (7 mm thick; e.g. Chemglass Inc. # CG 201-51, coarse porosity) by adding excess synthetic urine on the top of the disc. Repeating until the disc is saturated. Place the saturated fritted disc in the hydrating dish and add the synthetic urine until it reaches the level of the disc. The fluid height must not exceed the height of the disc.

Place the collection reservoir on the balance and connect the digital output of the balance to a computerized data acquisition system. Position the ring stand with a 16 mesh rigid stainless steel support screen above the collection dish. This 16 mesh screen should be sufficiently rigid to support the SFC apparatus during the measurement. The support screen must be flat and level.

### AGM Sampling

AGM samples should be stored in a closed bottle and kept in a constant, low humidity environment. Mix the sample to evenly distribute particle sizes. Remove a representative sample of material to be tested from the center of the container using the spatula. The use of a sample divider is recommended to increase the homogeneity of the sample particle size distribution.

### SFC Procedure

Position the weighing funnel on the analytical balance plate and zero the balance. Using a spatula weigh 0.9 g (±0.05 g) of AGM into the weighing funnel. Position the SFC cylinder on the bench, take the weighing funnel and gently, tapping with finger, transfer the AGM into the cylinder being sure to have an evenly dispersion of it on the screen. During the AGM transfer, gradually rotate the cylinder to facilitate the dispersion and get homogeneous distribution. It is important to have an even distribution of particles on the screen to obtain the highest precision result. At the end of the distribution the AGM material must not adhere to the cylinder walls. Insert the plunger shaft into the lid central hole then insert the plunger center into the cylinder for few centimeters. Keeping the plunger center away from AGM insert the lid in the cylinder and carefully rotate it until the alignment between the two is reached. Carefully rotate the plunger to reach the alignment with lid then move it down allowing it to rest on top of the dry AGM. Insert the stainless steel weight to the plunger rod and check if the lid moves freely. Proper seating of the lid prevents binding and assures an even distribution of the weight on the gel bed.

The thin screen on the cylinder bottom is easily stretched. To prevent stretching, apply a sideways pressure on the plunger rod, just above the lid, with the index finger while grasping the cylinder portion of the apparatus. This “locks” the plunger in place against the inside of the cylinder so that the apparatus can be lifted. Place the entire apparatus on the fritted disc in the hydrating dish. The fluid level in the dish should not exceed the height of the fritted disc. Care should be taken so that the layer does not loose fluid or take in air during this procedure. The fluid available in the dish should be enough for all the swelling phase. If needed, add more fluid to the dish during the hydration period to ensure there is sufficient synthetic urine available. After a period of 60 minutes, place the SFC apparatus under the caliper gauge and record the caliper as L2 to the nearest of 0.01 mm. Calculate, by difference L2−L1, the thickness of the gel layer as L0 to the nearest ±0.1 mm. If the reading changes with time, record only the initial value.

Transfer the SFC apparatus to the support screen above the collection dish. Be sure, when lifting the apparatus, to lock the plunger in place against the inside of the cylinder. Position the constant hydrostatic head reservoir such that the delivery tube is placed through the hole in the cylinder lid. Initiate the measurement in the following sequence:
a) Open the stopcock of the constant hydrostatic head reservoir and permit the fluid to reach the 5 cm mark. This fluid level should be obtained within 10 seconds of opening the stopcock.
b) Once 5 cm of fluid is attained, immediately initiate the data collection program.

With the aid of a computer attached to the balance, record the quantity of fluid passing through the gel layer versus time at intervals of 20 seconds for a time period of 10 minutes. At the end of 10 minutes, close the stopcock on the reservoir. The data from 60 seconds to the end of the experiment are used in the calculation. The data collected prior to 60 seconds are not included in the calculation. Perform the test in triplicate for each AGM sample.

Evaluation of the measurement remains unchanged from EP-A 640 330. Through-flux is captured automatically.

Saline flow conductivity (SFC) is calculated as follows:

SFC [cm³s/g]=(Fg(t=0)×L₀)/(d×A×WP),

where Fg(t=0) is the through-flux of NaCl solution in g/s, which is obtained from a linear regression analysis of the Fg(t) data of the through-flux determinations by extrapolation to t=0, L₀ is the thickness of the gel layer in cm, d is the density of the NaCl solution in g/cm³, A is the area of the gel layer in cm² and WP is the hydrostatic pressure above the gel layer in dyn/cm².

### Particle Size Distribution

Particle size distribution is determined by the EDANA (European Disposables and Nonwovens Association) recommended test method No. 420.2-02 “Particle Size Distribution”.

### 16 h Extractables

The level of extractable constituents in the water-absorbing polymeric particles is determined by the EDANA (European Disposables and Nonwovens Association) recommended test method No. 470.2-02 “Determination of extractable polymer content by potentiometric titration”. Extraction time is 16 hours.

### pH Value

The pH of the water-absorbing polymeric particles is determined by the EDANA (European Disposables and Nonwovens Association) recommended test method No. 400.2-02 “Determination of pH”.

### Free Swell Rate (FSR)

1.00 g (=W1) of the dry water-absorbing polymeric particles is weighed into a 25 ml glass beaker and is uniformly distributed on the base of the glass beaker. 20 ml of a 0.9% by weight sodium chloride solution are then dispensed into a second glass beaker, the contents of this beaker are rapidly added to the first beaker and a stopwatch is started. As soon as the last drop of salt solution is absorbed, confirmed by the disappearance of the reflection on the liquid surface, the stopwatch is stopped. The exact amount of liquid poured from the second beaker and absorbed by the polymer in the first beaker is accurately determined by weighing back the second beaker (=W2).

The time needed for the absorption, which was measured with the stopwatch, is denoted t. The disappearance of the last drop of liquid on the surface is defined as time t.

The free swell rate (FSR) is calculated as follows:

FSR [g/gs]=W2/(W1×t)

When the moisture content of the base polymer is more than 3% by weight, however, the weight W1 must be corrected for this moisture content.

Surface tension of aqueous extract (STR=Surface Tension Reduction)

0.50 g of the water-absorbing polymeric particles is weighed into a small glass beaker and admixed with 40 ml of 0.9% by weight salt solution. The contents of the beaker are magnetically stirred at 500 rpm for 3 minutes and then allowed to settle for 2 minutes. Finally, the surface tension of the supernatant aqueous phase is measured with a K10-ST digital tensiometer or a comparable apparatus having a platinum plate (from Kruess). The measurement is carried out at a temperature of 23° C.

### Moisture Content of Base Polymer

The water content of the water-absorbing polymeric particles is determined by the EDANA (European Disposables and Nonwovens Association) recommended test method No. 430.2-02 “Moisture content”.

### Base Polymer (not According to the Invention)

The commercial product ASAP 510 Z (surface crosslinked) with a broad particle size distribution (150-850 μm) from BASF AG was employed in the following examples according to the invention and coated in a continuous fluidized bed reactor.

### EXAMPLE 1

A continuous fluidized bed unit on a pilot plant scale having a rectangular inflow surface of 0.5 m² was used. Nitrogen at a temperature of approx. 24° C. with an inflow velocity of 1.2 m/s was used as carrier gas. The fluidized bed unit was equipped with 4 two-fluid nozzles having an aperture diameter of 2 mm mounted close to the bottom. The atomizer gas was nitrogen at a temperature of 21° C.

30 kg of the absorbent polymer (ASAP 510 Z in this case) were loaded in advance into this fluidized bed unit. At the front section of the unit the absorbent polymer was fed in continuously at the rate of approx. 100 kg/h and taken off at the opposite weir barrier.

An aqueous dispersion composed of calcium phosphate and polyol TP 70 (sevenfold ethoxylated tris(hydroxymethyl)propane from Perstorp) at a temperature of 21° C. was sprayed on at the rate of 5 kg/h. In this way 0.5% by weight of calcium phosphate and 0.4% by weight of polyol TP 70 (each with respect to the amount of absorbent polymer employed) was applied to the surface of the absorbent polymer.

The coated material was taken off at the discharge point and lumps were removed by means of a coarse sieve (1,000 μm). The application-related properties of the water-absorbent material are presented in Table 1.

### EXAMPLE 2

The procedure was completely analogous to Example 1. At variance with Example 1 an aqueous aluminum sulfate solution was sprayed on via the two-fluid nozzles close to the bottom at a mass flow rate of approx. 5 kg/h. Altogether 0.2% by weight of aluminum sulfate (calculated as 100% aluminum sulfate) was applied to the surface of the absorbent polymer. (The stated percentage by weight relates to the absorbent polymer employed.)

The coated material was taken off at the discharge point and lumps were removed by means of a coarse sieve (1,000 μm). The application-related properties of the water-absorbent material are presented in Table 1.

### EXAMPLE 3

The procedure was completely analogous to Example 1. At variance with Example 1, air was used as carrier gas and an aqueous dispersion of calcium phosphate and polyol TP 70 (sevenfold ethoxylated tris(hydroxymethyl)propane from Perstorp) at a temperature of 21° C. was sprayed on via the two-fluid nozzles close to the bottom at a mass flow rate of approx. 5 kg/h. In this way 0.5% by weight of calcium phosphate and 0.2% by weight of polyol TP 70 (each with respect to the absorbent polymer employed) was applied to the surface of the absorbent polymer.

The coated material was taken off at the discharge point and lumps were removed by means of a coarse sieve (1,000 μm). The application-related properties of the water-absorbent material are presented in Table 1.

### EXAMPLE 4

The procedure was completely analogous to Example 1. At variance with Example 1, air was used as carrier gas and an aqueous aluminum sulfate solution was sprayed on via the two-fluid nozzles close to the bottom at a mass flow rate of approx. 5 kg/h. In this way 0.1% by weight of aluminum sulfate (calculated as 100% aluminum sulfate) was applied to the surface of the absorbent polymer. Percentages by weight relate to the absorbent polymer employed.

The coated material was taken off at the discharge point and lumps were removed by means of a coarse sieve (1,000 μm). The application-related properties of the water-absorbent material are presented in Table 1.

TABLE 1

| | CRC | AUL 0.7 psi | SFC |
|---|---|---|---|
| | [g/g] | [g/g] | [×10⁻⁷ cm³ s/g] |
| Base polymer | 27.7 | 23.9 | 51 |
| ASAP 510 Z | | | |
| Example 1 | 27.6 | 24.0 | 102 |
| Example 2 | 27.4 | 22.0 | 94 |
| Example 3 | 27.8 | 23.8 | 99 |
| Example 4 | 27.5 | 22.8 | 75 |

### Base Polymer (not According to the Invention)

The commercial product ASAP 500 Z (surface crosslinked) with a broad particle size distribution (150-850 μm) of which the particles smaller than 150 μm have been removed and a pH of 5.75 from BASF AG was employed in the following examples according to the invention and coated in a continuous fluidized bed reactor.

### EXAMPLE 5

The procedure was completely analogous to Example 1. At variance with Example 1, air was used as carrier gas and ASAP 500 Z was used as polymer.

An aqueous dispersion of calcium phosphate and polyol TP 70 (sevenfold ethoxylated tris(hydroxymethyl)propane from Perstorp) at a temperature of 21° C. was sprayed on via the two-fluid nozzles close to the bottom at a mass flow rate of approx. 5 kg/h. In this way 0.5% by weight of calcium phosphate and 0.2% by weight of polyol TP 70 (each with respect to the absorbent polymer employed) was applied to the surface of the absorbent polymer. The calciumphosphate has actually been dispersed in water and the respective amount of Polyol TP 70 has been dissolved in this aqueous dispersion.

The coated material was taken off at the discharge point and lumps were removed by means of a coarse sieve (1,000 μm). The application-related properties of the water-absorbent material are presented in Table 2.

The other material properties are as follows:

### Flowrate=8.7 g/s

Apparent Bulk Density=0.62 g/ml
Residual acrylic acid monomer=268 ppm
Extractables 16 h=9.4 wt. %
Surface tension of aqueous extract (STR)=69 mN/m

### L-color=89.7

a-color=−0.25
b-color=4.0

Particle size distribution:

>850 μm=0.2 wt. %
600-850 μm=32 wt. %
300-600 μm=52 wt. %
150-300 μm=15 wt. %
45-150 μm=0.7 wt. %
<45 μm=<0.1 wt. %

### EXAMPLE 6

The procedure was completely analogous to Example 1. At variance with Example 1, air was used as carrier gas and ASAP 500 Z was used as polymer.

An aqueous dispersion of calcium phosphate and Glycerine at a temperature of 21° C. was sprayed on via the two-fluid nozzles close to the bottom at a mass flow rate of approx. 5 kg/h. In this way 0.5% by weight of calcium phosphate and 0.2% by weight of Glycerine (each with respect to the absorbent polymer employed) was applied to the surface of the absorbent polymer. The calciumphosphate has actually been dispersed in water and the respective amount of Glycerine has been dissolved in this aqueous dispersion.

The coated material was taken off at the discharge point and lumps were removed by means of a coarse sieve (1,000 μm). The application-related properties of the water-absorbent material are presented in Table 2.

### EXAMPLE 7

The procedure was completely analogous to Example 1. At variance with Example 1, air was used as carrier gas and ASAP 500 Z was used as polymer.

An aqueous dispersion of calcium phosphate and Gly-7 EO (sevenfold ethoxylated glycerine) at a temperature of 21° C. was sprayed on via the two-fluid nozzles close to the bottom at a mass flow rate of approx. 5 kg/h. In this way 0.5% by weight of calcium phosphate and 0.2% by weight of Gly-7 EO (each with respect to the absorbent polymer employed) was applied to the surface of the absorbent polymer. The calciumphosphate has actually been dispersed in water and the respective amount of Gly-7 EO has been dissolved in this aqueous dispersion.

The coated material was taken off at the discharge point and lumps were removed by means of a coarse sieve (1,000 μm). The application-related properties of the water-absorbent material are presented in Table 2.

TABLE 2

| | CRC | AUL 0.7 psi | FSR | SFC |
|---|---|---|---|---|
| | [g/g] | [g/g] | [g/g · s] | [×10⁻⁷ cm³ s/g] |
| Base polymer | 30.0 | 24.0 | 0.15 | 28 |
| ASAP 500 Z | | | | |
| Example 5 | 30.1 | 24.3 | 0.20 | 50 |
| Example 6 | 29.9 | 24.5 | 0.18 | 61 |
| Example 7 | 30.3 | 24.1 | 0.19 | 49 |

1-18. (canceled)

19. A process for producing a water-absorbing material comprising spray-coating water-absorbing polymeric particles with at least one non-reactive coating agent in a continuous process in a fluidized bed reactor at a temperature in the range of from 0° C. to 150° C., with the proviso that said non-reactive coating agents do not comprise an elastic film-forming polymer.

20. A process for producing a water-absorbing material comprising spray-coating water-absorbing polymeric particles with at least one non-reactive coating agent in a continuous process in a fluidized bed reactor at a temperature in the range of from 0° C. to 150° C., wherein said non-reactive coating agent is selected from the group consisting of water-insoluble inorganic powders, water-soluble multivalent metal salts, polycationic polymers, sawdust, and binding agents.

21. The process of claim 19, wherein said water-absorbing polymeric particles are post-crosslinked.

22. The process of claim 19, wherein said non-reactive coating agent is a water-insoluble inorganic powder and/or a water-soluble multivalent metal salt.

23. The process of claim 19, wherein said water-insoluble inorganic powder is applied in an amount in the range of from 0.001% to 20% by weight based on the weight of the water-absorbing polymeric particles.

24. The process of claim 19, wherein said non-reactive coating agent is sawdust and optionally a binding agent.

25. The process of claim 19, wherein said non-reactive coating agent is a water-insoluble inorganic powder together with a binding agent.

26. The process of claim 19, wherein said water-absorbing polymeric particles are spray-coated with an aqueous dispersion of the water-insoluble salt.

27. The process of claim 19, wherein said coating is applied in a coater using the Wurster- or Glatt-Zeller principles.

28. The process of claim 19, wherein said water-absorbing polymeric particles are spray-coated at a product and/or carrier gas temperature in the range of from 0 to 120° C.

29. The process of claim 28, wherein the gas stream in said fluidized bed reactor is selected such that the relative moisture at the point of exit of said gas stream is in the range of from 0.1% to 90%.

30. The process of claim 19, wherein said coating is carried out under inert gas.

31. A water-absorbing material prepared by the process of claim 19.

32. A water-absorbing material prepared by the process of claim 24.

33. The process of claim 24, wherein said coating is an anti-microbial coating.

34. A hygiene article or packaging material comprising the water-absorbing polymeric material of claim 31.

35. A baby diaper or incontinence product comprising the water-absorbing material of claim 31.
